(19) United States
(12) Patent Application Publication
(10) Pub. No.: US 2019/0352242 A1
(43) Pub. Date: Nov. 21, 2019

(54) SYSTEMS AND METHODS FOR SEPARATING (E)-1-CHLORO-3,3,3-TRIFLUOROPROPENE, HF, AND A HEAVY ORGANIC AND REACTOR PURGE

(71) Applicant: Honeywell International Inc., Morris Plains, NJ (US)

(72) Inventors: Yuon Chiu, Denville, NJ (US); Stephen A. Cottrell, Baton Rouge, LA (US); Gustavo Cerri, Parsippany, NJ (US); Jennifer W. McClaine, Branchburg, NJ (US); Tao Wang, Shanghai (CN); Rajiv Ratna Singh, Getzville, NY (US); Rajiv Banavali, Morristown, NJ (US)

(21) Appl. No.: 16/529,287

(22) Filed: Aug. 1, 2019

Related U.S. Application Data
(63) Continuation of application No. 15/850,724, filed on Dec. 21, 2017, now abandoned.
(60) Provisional application No. 62/443,349, filed on Jan. 6, 2017.

(51) Int. Cl. C07C 17/386; B01D 3/14; B01D 3/36; C07C 19/01; C07C 21/18; C07C 17/38; B01D 3/00; C01B 7/19; C07C 17/383; B01D 3/06

(52) U.S. Cl. CPC C07C 17/386; B01D 3/143; B01D 3/36; C07C 19/01; C07C 21/18; C07C 17/38; B01D 2257/70; C01B 7/196; C07C 17/383; B01D 3/06; Y02P 20/127; B01D 2257/2047; B01D 3/009

(57) ABSTRACT

The present disclosure provides separation processes for removing heavy organics that are formed in various production processes of HCFO-1233zd(E). Such separation processes allow for the recovery and/or separation of the heavy organics from reactants that are used to form HCFO-1233zd(E), including HF. Such separation or recovery processes may utilize various separation techniques (e.g., decanting, liquid-liquid separation, distillation, and flash distillation) and may also utilize the unique properties of azeotropic or azeotrope-like compositions. Recovery of the heavy organic that is substantially free from HF may allow for their use in subsequent manufacture processes or disposal.

### CROSS REFERENCE TO RELATED APPLICATIONS

This application is a Continuation of U.S. patent application Ser. No. 15/850,724, entitled SYSTEMS AND METHODS FOR SEPARATING (E)-1-CHLORO-3,3,3-TRIFLUOROPROPENE, HF, AND A HEAVY ORGANIC AND REACTOR PURGE, filed on Dec. 21, 2017 which claims the benefit under Title 35, U.S.C. § 119(e) of U.S. Provisional Patent Application Ser. No. 62/443,349, entitled SYSTEMS AND METHODS FOR SEPARATING (E)-1-CHLORO-3,3,3-TRIFLUOROPROPENE, HF, AND A HEAVY ORGANIC AND REACTOR PURGE, filed on Jan. 6, 2017, the entire disclosures of which are expressly incorporated by reference herein.

### FIELD OF THE DISCLOSURE

This disclosure relates the separation of HF from heavy organics. More specifically, this disclosure relates to the separation and recovery of heavy organics from the production of ((E)-1-chloro-3,3,3-trifluoropropene (HCFO-1233zd(E)).

### BACKGROUND

Fluorocarbon based fluids have found widespread use in industry in a number of applications, including use as refrigerants, aerosol propellants, blowing agents, heat transfer media, and gaseous dielectrics. Due to suspected environmental problems associated with the use of some of these fluids, including the relatively high global warming potentials associated therewith, it is desirable to use fluids having the lowest possible global warming potential (GWP) in addition to also having zero ozone depletion potential (ODP). Thus, there is considerable interest in developing environmentally friendlier materials for the applications mentioned above.

Hydrochlorofluoroolefins (HCFOs) having zero ozone depletion and low global warming potential have been identified as potentially filling this need. However, the toxicity, boiling point, and other physical properties of such chemicals vary greatly from isomer to isomer. One HCFO having valuable properties is (E)-1-chloro-3,3,3-trifluoropropene (HCFO-1233zd(E)), which has been proposed as a next generation non ozone depleting and low global warming potential solvent.

The processes for the manufacture of HCFO-1233zd(E) produces various by-products, such as various heavy organics. Furthermore, HCFC-1233zd(Z) and HCFC-244fa are also intermediates in the production of HCFO-1233zd(E), as described in U.S. Pat. Nos. 7,829,747, 8,217,208, 8,835,700, and 9,045,386, the disclosures of which are incorporated herein by reference.

As used herein, the term “heavy organic(s)” or “heavy organic(s) phase” may include tar or tar-like substances, or oligomers formed from the production of HCFO-1233zd(E). The term “heavy organic(s)” may be understood to be organic compositions (e.g., chains of C, H, O, F, Cl, etc., and combinations thereof) having a weight average molecular weight (Mw) between about 500 g/mol to about 7,000 g/mol. For example, the heavy organics may have a molecular weight as little as 500 g/mol, 550 g/mol, 590 g/mol, 600 g/mol, 800 g/mol, 1,000 g/mol, as great as 1,200 g/mol, 3,000 g/mol, 4,000 g/mol, 5,000 g/mol, and 6,000 g/mol, 7,000 g/mol or within any range defined between any two of the foregoing values, for example, such as 500 g/mol to 700 g/mol, from 600 g/mol to 6,000 g/mol, and from 1,000 g/mol to 1,200 g/mol.

Furthermore, the term “heavy organic(s)” may be understood to include organic compounds composed of single units or monomers, may comprise various comonomers, and may have a degree of polymerization between and including 1 to 15. For example, the degree of polymerization may be as little as 1, 2, 4, 5, or as great as 9, 10, 12, 15, or within any range defined between any two of the foregoing values, such as 1 to 15, 2 to 12, 4 to 10 and 5 to 9, for example, and including the endpoints (e.g., between and including 1 to 15, between and including 2 to 10, and between and including 5 to 9).

In various embodiments, the heavy organic may have a boiling point between about 120° C. and about 300° C. at a pressure between about 3 psia to about 73 psia. The boiling point may be as little as about 60° C., 80° C., 100° C., as great as 350° C., 400° C., 500° C., or within any range defined between any two of the fore going values (e.g., between about 60° C. and about 500° C.).

Because the boiling points of HCFO-1233zd(E) and other reactant/products including HCFO-1233zd(Z), 1,1,1,3,3 pentachloropropane (240fa), 1,1,1,3 tetrachoro-3 fluoro-propane (241fa), 1,1,1-trichloro-3,3-difluoro-propane (242fa) are similar and many intermolecular forces are present, conventional separation techniques can prove somewhat difficult to accomplish. Furthermore, because some azeotropes and/or heteroazeotropes can be formed between various combinations of the aforementioned compounds, effective separation of the aforementioned compounds from the heavy organic are needed.

Also, because HF is an effective solvent, efficient removal of HF from the heavy organics is desired. Because HF must be removed from the heavy organics before the heavy organics may be utilized in subsequent processes or disposed of, a need therefore exists to address separation of the heavy organics in a purge stream from a reactor producing 1233zd(E) from other compounds, including HF.

### SUMMARY

The present disclosure provides separation processes for heavy organics that result from various production processes of HCFO-1233zd(E). Such separation processes allow for the recovery and/or separation of the heavy organics from reactants needed to form HCFO-1233zd(E), including HF. Such separation or recovery processes may utilize various separation techniques (e.g., decanting, liquid-liquid separation, distillation, and flash distillation) and may also utilize the unique properties of azeotropic or azeotrope-like compositions. Recovery of the heavy organics that are substantially free from HF may allow for their use in subsequent manufacture processes or disposal.

Methods of cleaning a reactor may include removing a reactor purge containing HF and a heavy organic, separating an HF phase and an organic phase comprising (E)-1-chloro-3,3,3-trifluoropropene and the heavy organic, distilling the heavy organic phase, and recovering the distilled heavy organic. In various embodiments, the separating the HF phase and the organic phase may include at least one of decanting, centrifuging, liquid-liquid extraction, distilling, flash distilling, crystallization/filtration, or combinations thereof. As used herein, the types of distillation are not particularly limited and may include, for example, simple distillation, molecular distillation, vacuum distillation, batch distillation, continuous distillation, flash distillation, fractional distillation, azeotropic distillation, and combinations thereof.

In various embodiments, the separation of HF and the heavy organic may be done at a higher pressure, a higher temperature, or both a higher pressure and temperature than the reactor purge when recovered. In some embodiments, the separation may be done at a lower temperature or lower pressure, or both a lower pressure and a lower pressure than the reactor purge when recovered.

In some embodiments, an azeotropic or azeotrope-like composition may be formed. The azeotropic or azeotrope-like composition may include an azeotrope between HF and at least one of 240, 241, 242, or combinations thereof. In some embodiments the azeotropic or the azeotrope-like composition may comprise a heteroazeotrope. The azeotropic or azeotrope-like composition may have a boiling point of about 0° C. to about 60° C. at a pressure of about 3 psia to about 73 psia.

Methods of separating (E)-1-chloro-3,3,3-trifluoropropene, HF, and a heavy organic, may include the steps of providing a mixture of (E)-1-chloro-3,3,3-trifluoropropene, HF, and the heavy organic to a liquid-liquid separator, separating an HF phase and an organic phase comprising (E)-1-chloro-3,3,3-trifluoropropene and heavy organic, distilling the HF phase to form an HF rich overhead and a light organics bottoms, adding a light organics phase to the liquid-liquid separator, distilling the heavy organic from the liquid-liquid separator, and recovering the heavy organic.

Methods may also include adding a washing fluid to the mixture of (E)-1-chloro-3,3,3-trifluoropropene, HF, and heavy organic. The washing fluid may include at least one of 1-chloro-3,3,3-trifluoropropene, 1,1,1,3,3-pentachloropropane, 1,1,1,3-tetrachoro-3-fluoro-propane, 1,1,1 trichloro-3,3-difluoro-propane, HCl, or mixtures thereof.

The separating the HF phase and the organic phase may include at least one of decanting, centrifuging, liquid-liquid extraction, distilling, flash distilling, or combinations thereof.

Furthermore, various methods may also include or comprise recovering the light organics after the distilling the organic phase from the liquid-liquid separator and/or condensing the mixture of (E)-1-chloro-3,3,3-trifluoropropene, HF, and the heavy organic.

Methods may also include forming an azeotropic or an azeotrope-like composition. The azeotropic or the azeotrope-like composition includes an azeotrope between HF and at least one of 240, 241, 242, or combinations thereof. The azeotrope-like composition may be a homogeneous azeotrope or a heteroazeotrope.

### BRIEF DESCRIPTION OF THE DRAWINGS

The above mentioned and other features and objects of this disclosure, and the manner of attaining them, will become more apparent and the disclosure itself will be better understood by reference to the following description of exemplary embodiments of the disclosure taken in conjunction with the accompanying drawings, wherein:

FIG. 1A is a process flow diagram showing the processing of reactor purge resulting from the production of HCFO-1233zd(E);

FIG. 1B is a process flow diagram similar to FIG. 1A, showing the processing of reactor purges from a plurality of reactors resulting from the production of HCFO-1233zd(E);

FIG. 1C is a process flow diagram showing the processing of reactor purge resulting from the production of HCFO-1233zd(E) where the HF Overhead of the organics phase is recycled back to the reactor;

FIG. 2A and FIG. 2B are a process flow diagrams showing flash-distillation processing of reactor purge resulting from the production of HCFO-1233zd(E);

FIG. 3 is a process flow diagram showing the processing of reactor purge including adding a washing fluid;

FIG. 4 is yet another process flow diagram showing a process where the overhead of the distilled HF phased is further separated according to various embodiments; and

FIG. 5 is a process flow diagram showing the processing of reactor purge including adding a washing fluid and decanting the HF phase according to various embodiments.

Corresponding reference characters indicate corresponding parts throughout the several views. Although the drawings represent embodiments of the present disclosure, the drawings are not necessarily to scale and certain features may be exaggerated in order to better illustrate and explain the present disclosure. The exemplification set out herein illustrates exemplary embodiments of the disclosure, in various forms, and such exemplifications are not to be construed as limiting the scope of the disclosure in any manner.

### DETAILED DESCRIPTION

As briefly described above, this disclosure provides for separation and recovery techniques of HF and light organics from heavy organics that are produced during the production of (E)-1-chloro-3,3,3-trifluoropropene (HCFO-1233zd(E)). Separation and recovery of the heavy organics that are substantially free of HF is desirable because it will allow for either the utilization of the heavy organics in subsequent processes, alternative uses, or may allow for the disposal of the heavy organics in a relatively cost effective and environmentally friendly manner.

Waste streams or purge streams from reactors producing (E)-1-chloro-3,3,3-trifluoropropene (HCFO-1233zd(E)) often include various compounds, including but not limited to 1,1,1,3,3 pentachloropropane (240fa), 1,1,1,3 tetrachoro-3 fluoro-propane (241fa), 1,1,1-trichloro-3,3-difluoro-propane (242fa), HF, HCl, HCFO-1233zd(E), and various heavy organics. Separation of HF and other materials can prove somewhat difficult to separate with conventional separation techniques because of the solvent properties of HF, azeotropic or azeotrope-like mixtures that may form, and subsequent reaction during separation. Thus, disclosed below are various examples or embodiments of methods that allow for the separation of HF and other materials from the heavy organics.

As used herein, the modifier “about” used in connection with a quantity is inclusive of the stated value and has the meaning dictated by the context (for example, it includes at least the degree of error associated with the measurement of the particular quantity). When used in the context of a range, the modifier “about” should also be considered as disclosing the range defined by the absolute values of the two endpoints. For example, the range “from about 2 to about 4” also discloses the range “from 2 to 4.”

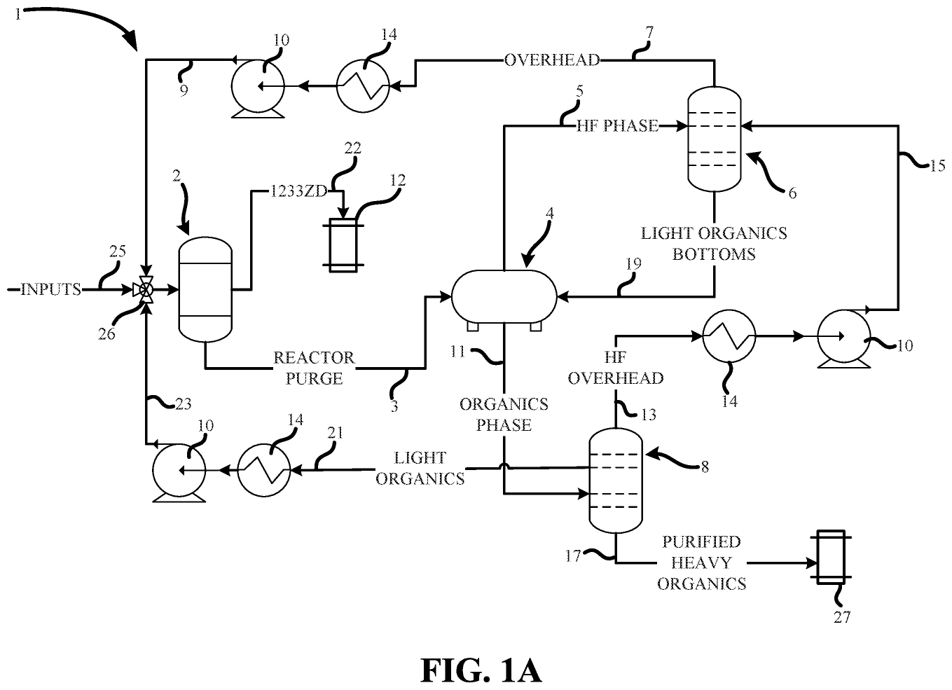

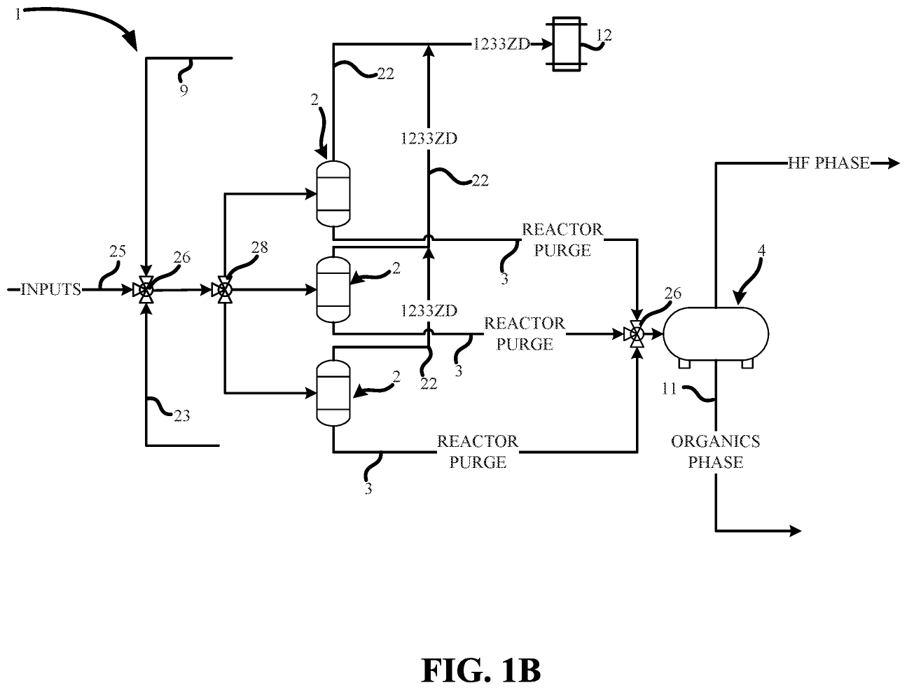

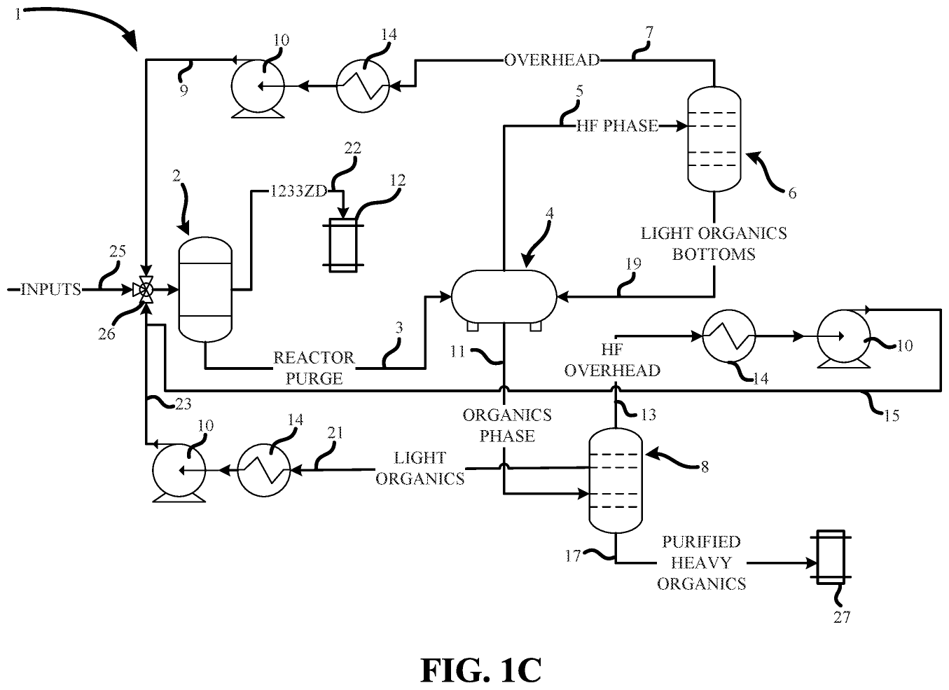

FIG. 1A illustrates a process flow diagram illustrating process flow 1 according to various embodiments. Process flow 1 illustrates the inputs or reactants stream 25 flowing into reactor 2 for the production of 1233zd. The processing parameters for the production of 1233zd are not particularly limited and may include any known process for producing 1233zd. For example, processes for HCFC-1233zd production are detailed in U.S. Pat. Nos. 8,921,621 and 8,835,770, the disclosures of which are both herein incorporated by reference in their entirety.

The produced 1233zd(E) may flow via 1233zd stream 22 to 1233zd container 12 to be collected, purified, and shipped in 1233zd container 12. The reactor may also have a purge stream 3 that removes the purge material from the reactor 2. The purge stream 3 is not particularly limited and may be operated on a continually, semi-continually, or using a batch method.

Furthermore, the purge stream 3 may be a combination of purge streams from various reactors. For example, with temporary reference to FIG. 1B, a portion of process flow 1 is illustrated with a plurality of reactors. In FIG. 1B, three reactors 2 are shown. The reactants stream 25 may be combined with HF recycle stream 9 and light organics recycle stream 23 in input valve 26. Distributor valve 28 may then distribute the stream from input valve 26.

Without being limited to any particular embodiment, the incorporation of multiple reactors in parallel may allow for the shutdown and/or cleaning of one reactor while the remaining reactors continue to operate. Thus, 1233zd(E) may be produced on a continual basis or may be produced on a batch basis from the remaining reactors while a reactor is out of operation for maintenance and/or cleaning. In such embodiments, it is believed that a more consistent and predictable supply chain may be achieved, resulting in continual 1233zd(E) production capacity or near-continual 1233zd(E) production capacity.

With reference back to FIG. 1A, reactor 2 may also have a purge stream 3. Purge stream 3 is not particularly limited and may be continuously operated, semi-continuously operated, or operated as part of a batch process. In various embodiments, such as the embodiment shown in FIG. 1B, a plurality of purge streams 3 from a plurality of reactors 2 may be combined, for example with use of a variable valve 26.

The purge stream 3 may then be sent to separator 4. Separator 4 is not particularly limited and may be include at least one of decanting, centrifuging, liquid-liquid extraction, distilling, flash distilling, or combinations thereof. For example, as shown in FIG. 1B, separator 4 is illustrated as a liquid-liquid separator, where an HF rich phase is separated from an organics phase.

The amount of heavy organic material in overhead stream 5 may be less than 1 wt. %, or may be as little as 1 wt. %, 1.5 wt. %, or 2 wt. %, or may be as great as 5 wt. %, 6 wt. %, or 7 wt. %, or may be within any range defined between any two of the foregoing values, such as 1 wt. % to 7 wt. %, 1.5 wt. % to 6 wt. %, or 2 wt. % to 5 wt. %, for example. The amount of heavy organic material in bottoms stream 11 may be as little as 7 wt. %, 9 wt. %, or 11 wt. %, or may be as great as 15 wt. %, 20 wt. %, or 25 wt. %, or may be within any range defined between any two of the foregoing values, such as 7 wt. % to 25 wt. %, 9 wt. % to 20 wt. %, or 11 wt. % to 15 wt. %, for example.

HF overhead stream 5 is then sent to HF distillation column 6, where mainly HF and light organics are separated. HF rich overhead 7 is then sent to a condenser 14 and pump 10 and then forms part of HF recycle stream 9, which may then be recycled and incorporated in the production of 1233zd. Without being limited to any particular embodiment, it is believed that the use of recycled HF may help reduce production costs and reduce waste.

HF distillation column 6 may also have a light organics bottoms 19, which may then be sent back to separator 4. In various embodiments, light organics bottoms 19 may also have some amounts or traces of heavy organics. The light organics bottoms 11 may contain some traces of HF, light organics, and heavy organics. The light organics bottoms 11 may then be incorporated into the organics phase stream 11 and then sent to organics distillation column 8. Organics distillation column 8 may then separate HF, light organics, and heavy organics. HF distillation column 6 and organics distillation column 8 and other distillation columns may be understood to include—in some embodiments—characteristics, features, or sections common to conventional distillation columns. For example, distillation columns may include a rectifying section, a stripper section, a partial condenser, a partial vaporizer, or combinations thereof.

As used herein, the term “light organic(s)” may include various organic compositions (e.g., chains of C, H, O, F, Cl, and combinations thereof) having a weight average (Mw) molecular weight above about 50 g/mol to below about 450 g/mol, including reactants for the formation of 1233zd(E), but is not limited to only reactants or inputs for the production of 1233zd(E). Thus, light organics may be understood to include HCFO-1233zd(Z), 1,1,1,3,3 pentachloropropane (240fa), 1,1,1,3 tetrachoro-3 fluoro-propane (241fa), 1,1,1-trichloro-3,3-difluoro-propane (242fa).

For example, the light organics may have a molecular weight as little as about 50 g/mol, about 100 g/mol, about 125 g/mol, about 150 g/mol, about 175 g/mol, as great as about 200 g/mol, about 225 g/mol, about 300 g/mol, about 400 g/mol, about 450 g/mol or within any range defined between any two of the foregoing values, such as between about 50 g/mol to about 450 g/mol, between about 150 g/mol to about 400 g/mol, and between about 175 g/mol to about 300 g/mol.

The following experimental solubility information presented in Table 1 below may be used by a person of ordinary skill in the art to tailor the various operating conditions of separators disclosed herein based on the composition of the various streams being separated.

TABLE 1
Solubility of 242fa, 241fa, and HF:

| | Organics in | HF in | Organics in |
|---|---|---|---|
| | HF Phase | Organic Phase | HF Phase |
| | at 110° C. | at 110° C. | at 136° C. |
| Mixture | (wt. %) | (wt. %) | (wt. %) |
| 242fa/HF | 23 | 15 | 29 |
| 241fa/HF | 10 | 2 | |

The HF overhead 13 may be cooled and/or condensed in either a cooler or condenser (e.g., condenser 14), pumped via pump 10 and sent to HF distillation column 6 via HF rich stream 15. In some embodiments, such as the one exemplified in FIG. 1C, HF rich stream 15 may be recycled directly to input valve 26 to be added to reactor 2. The light organics may be sent via light organics stream 21, condensed via condenser 14 and pumped via pump 10 as condensed light organics stream 23 to input valve 26 to be incorporated in further production of 1233zd(E) in reactor 2. Finally, heavy organics may be recovered in heavy organics container 27 from purified heavy organics bottoms 17.

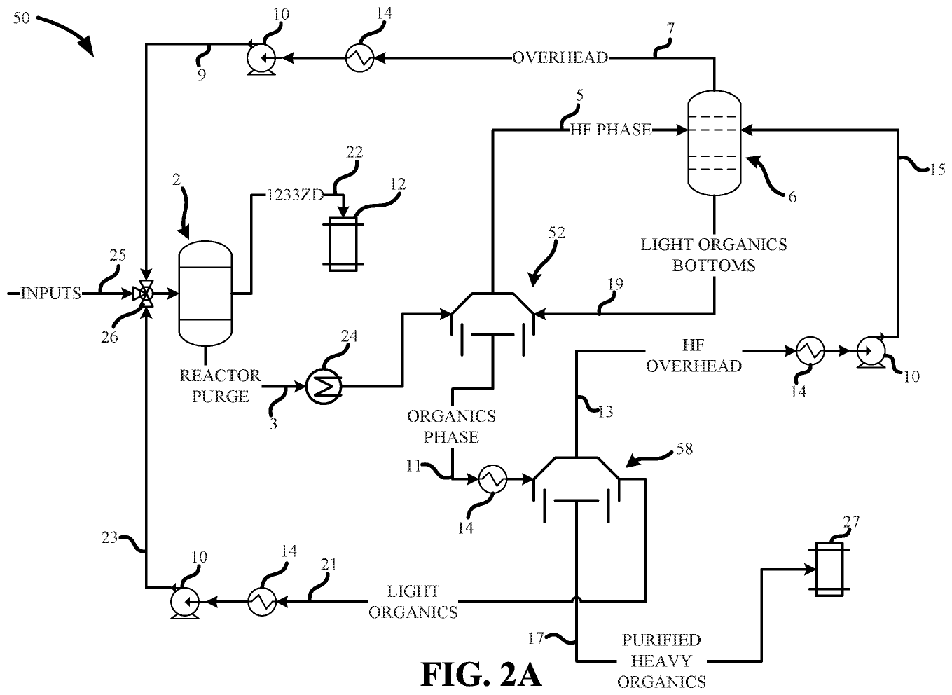

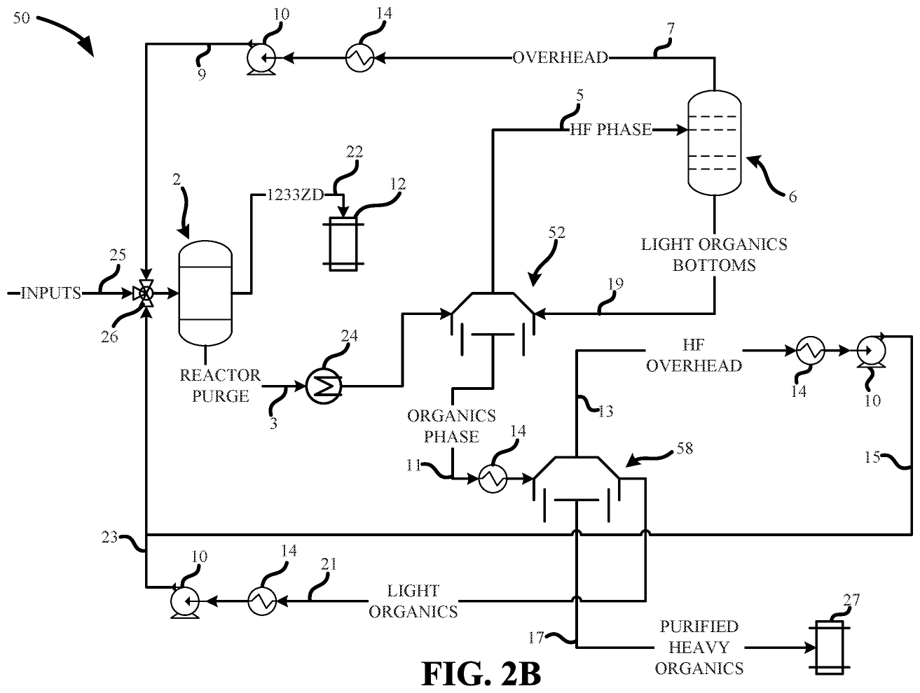

FIGS. 2A and 2B illustrate additional process flow diagrams for the production of 1233zd(E). Process 50, while somewhat similar to the processes shown in FIGS. 1A and 1B, incorporates the use of flash distillation at reduced temperatures and/or pressures. For example, reactor purge 3 from reactor 2 may be heated by pre-flash heat exchanger 24 and then sent to flash distillation separator 52. Flash distillation column is not particularly limited and may include any type of single stage or multi-stage flash distillation.

As used herein, flash distillation may be understood to include liquid feeds that pass through a heater or cooler (such as shown in FIGS. 2A and 2B as pre-flash heat exchanger 24) to cause the temperature of purge stream 3 to partially vaporize or vaporize. As the liquid/vapor of purge stream 3 from reactor 2 enters a reduced pressure vessel, the liquid and vapor separate. In various embodiments, because the vapor and liquid may be in such close contact up until the “flash”, or rapid separation, occurs, the product liquid and vapor phases may approach equilibrium. Moreover, as used herein, flash distillation may be understood to include pre-flashing, which may be used to reduce the load on flash distillation separator 52.

Without being limited to any theory, it is believed that in some embodiments, it is preferable to reduce the temperature and/or pressure of reactor bottoms stream 3 to prevent further chemical reactions downstream of reactor 2. Thus, by operating at a lower pressure downstream from cooling purge stream 3 from reactor 2, further undesirable reactions of chemicals contained in purge stream 3 (e.g., light organics and HF) may be reduced or eliminated.

Flash distillation separator 52 may then have an HF overhead stream 5, which may be sent to HF distillation column 6, and organics phase stream 11, which may be sent to organics flash distillation column 58. Organics flash distillation column 58 may then further separate HF, light organics, and the heavy organics. The HF overhead 13 may then be sent to HF distillation column 6. In some embodiments and as exemplified in FIG. 2B, HF overhead stream 5 may be sent to input valve 26 to be included as an input to reactor 2. The light organics stream 21 may then be recycled to input valve 26 and the purified heavy organics bottoms 17 may be sent to heavy organics container 27 for use in other processes or disposal.

The various separators, distillation columns, and flash distillation separators may be operated at various temperatures and pressures. Temperatures may range from as little as about −20° C., about 0° C., about 20° C., about 25° C., about 40° C., and as great as about 50° C., about 75° C., about 100° C., about 150° C., or within any range defined between any two of the foregoing values, for example, between about −20° C. to about 150° C., between about 0° C. to about 100° C., between about 20° C. to about 50° C.

Pressure may range from as little as about 2 psia, about 5 psia, about 10 psia, about 20 psia, as great as about 50 psia, about 100 psia, about 150 psia, about 300 psia, about 500 psia, about 550 psia, or within any range defined between any two of the foregoing values, for example between about 2 psia to about 500 psia, between about 5 psia to about 300 psia, and between about 10 psia to about 50 psia.

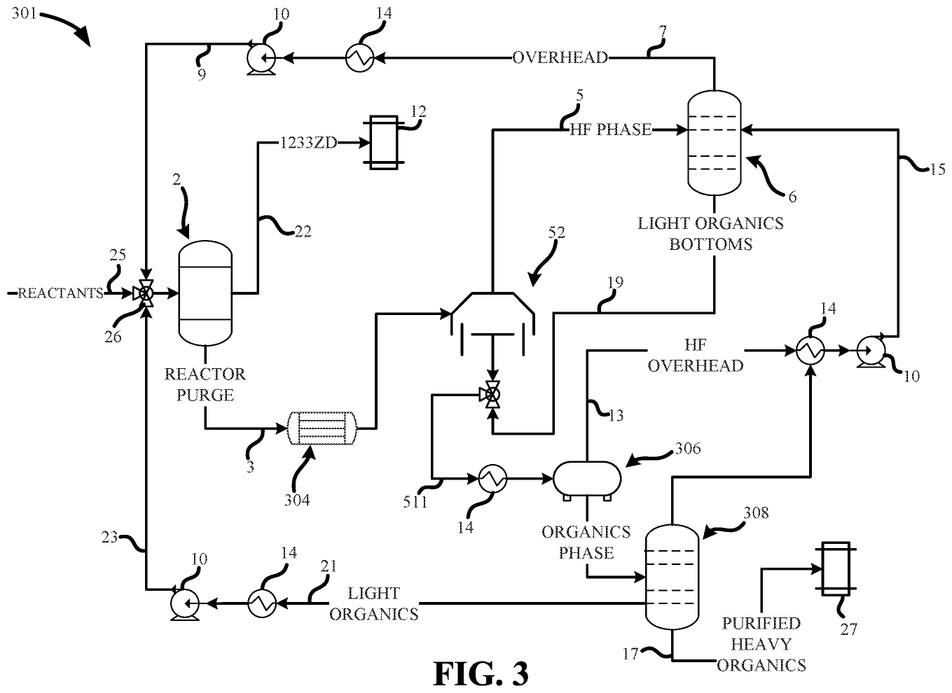

FIG. 3 illustrates yet another process flow diagram for process flow 301 with preconditioning. As used herein, the pre-conditioning is not particularly limited and may include any preconditioning known in separation processes. For example, in some processed, the reactor purge may be heated and partially flash distilled to reduce the HF load. Thus, by removing some HF from the mixture during the preconditioning process, the load on the downstream separation may be reduced. Reactor purge 3 may first be preconditioned by either altering the heat and/or pressure of reactor purge 3 (illustrated with pre-conditioner 304), and then first pre-flashing the reactor purge 3 in flash distillation separator 52. The bottoms, which may contain a higher phase of organics, may be combined with light organics bottoms stream 19 to form pre-conditioned stream 511. Pre-conditioned stream 511 may then be cooled and/or condensed in condenser 14 and send to liquid-liquid separator 306 to separate out the HF phase and the organic phase.

Without being limited to any theory, it is believed that in some embodiments, pre-conditioning the mixture may allow the separation process to be more effective, for example, with using various azeotropic or azeotrope-like mixtures.

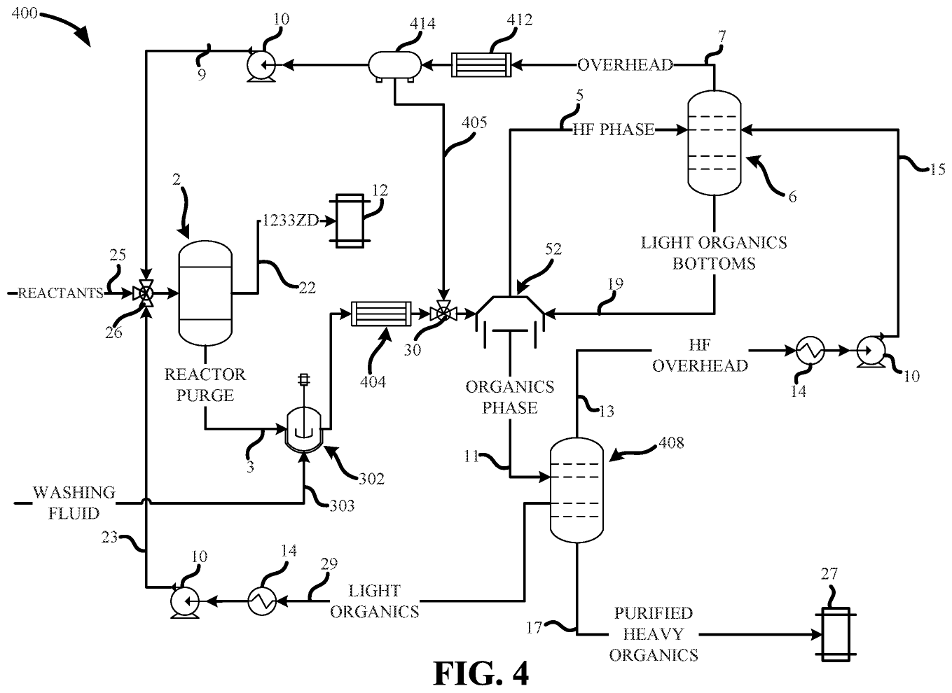

FIG. 4 illustrates a process flow diagram of yet another process using washing fluid 303. In the embodiment illustrated in FIG. 4, purge stream 3 from reactor 2 and washing fluid 303 are combined in mixer 302. As used herein the term washing fluid can be understood to be any fluid used to enrich or dilute a particular component of the mixture. For example, in some embodiments, the washing fluid may be a composition to enrich the light organics and increase their composition in the mixture. In some embodiments, it is preferable that washing fluid be other components found in reactant stream 25. However, it should be noted that the washing fluid source is not particularly limited and, in some embodiments, may include recycled components or compositions. For example, in some embodiments, the washing fluid 303 may be taken from the light organics phase stream 29 from distillation column 408.

The mixture from mixer 302 is then condensed in condenser 404 and is sent to mixture valve 30, where the mixture from mixer 302 is combined with overhead organics phase stream 405 from liquid-liquid separator 414. The mixture from mixture valve 30 is then sent to flash distillation separator 52, where the HF phase is separated (illustrated as HF overhead stream 5) from the organics phase (illustrated as organics phase stream 11). After the HF overhead stream 5 is sent to distillation column 6, the HF rich overhead 7 may be condensed in condenser 412.

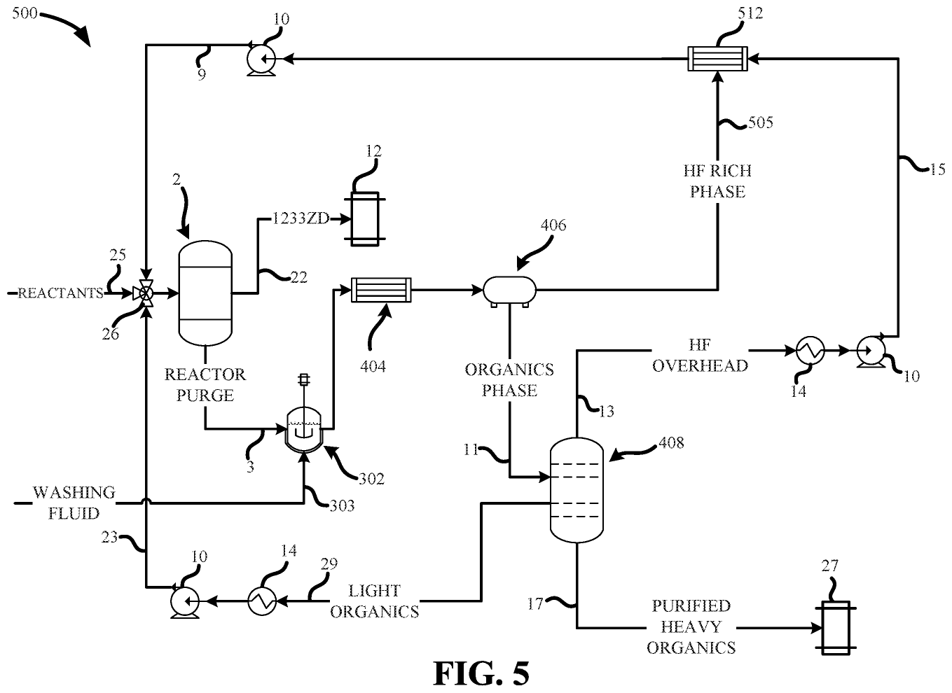

FIG. 5 illustrates another process similar to process flow 400 illustrated in FIG. 4, though with process flow 500, a liquid-liquid separator 406 is used to process the pre-conditioned reactor purge 3. HF rich phase 505 from liquid-liquid separator 406 may then be condensed in condenser 512 and recycled through pump 10 and form part of HF recycle stream 9 to be incorporated into the reactants for reactor 2. The organics phase stream 11 may then be sent to distillation column 408 to be further processed to separate HF, the light organics, and the heavy organics.

In the various processes described herein, separation may be facilitated by forming an azeotropic or azeotrope-like composition. The thermodynamic state of a fluid is defined by its pressure, temperature, liquid composition and vapor composition. For a true azeotropic composition, the liquid composition and vapor phase are essentially equal at a given temperature and pressure range. In practical terms this means that the components cannot be separated during a phase change. As disclosed herein, an azeotrope is a liquid mixture that exhibits a maximum or minimum boiling point relative to the boiling points of surrounding mixture compositions. Also, as used herein, the term “azeotrope-like” refers to compositions that are strictly azeotropic and/or that generally behave like azeotropic mixtures.

An azeotrope or an azeotrope-like composition is an admixture of two or more different components which, when in liquid form under a given pressure, will boil at a substantially constant temperature, which temperature may be higher or lower than the boiling temperatures of the individual components and which will provide a vapor composition essentially identical to the liquid composition undergoing boiling.

As used herein, azeotropic compositions may be defined to include azeotrope-like compositions, which is a composition that behaves like an azeotrope, i.e., that has constant boiling characteristics or a tendency not to fractionate upon boiling or evaporation. Thus, the composition of the vapor formed during boiling or evaporation is the same as or substantially the same as the original liquid composition. Hence, during boiling or evaporation, the liquid composition, if it changes at all, changes only to a minimal or negligible extent. This is in contrast with non-azeotrope-like compositions in which during boiling or evaporation, the liquid composition changes to a substantial degree.

Accordingly, the essential features of an azeotrope or an azeotrope-like composition are that at a given pressure, the boiling point of the liquid composition is fixed and that the composition of the vapor above the boiling composition is essentially that of the boiling liquid composition, i.e., essentially no fractionation of the components of the liquid composition takes place. Both the boiling point and the weight percentages of each component of the azeotropic composition may change when the azeotrope or azeotrope-like liquid composition is subjected to boiling at different pressures. Thus, an azeotrope or an azeotrope-like composition may be defined in terms of the relationship that exists between its components or in terms of the compositional ranges of the components or in terms of exact weight percentages of each component of the composition characterized by a fixed boiling point at a specified pressure.

In various embodiments of this disclosure, a composition which comprises effective amounts of HF, HCl, light organics, heavy organics, or combinations thereof to form an azeotropic or azeotrope-like composition is provided. As used herein, the term “effective amount” is an amount of each component which, when combined with the other component, results in the formation of an azeotrope or azeotrope-like mixture. As used herein, the terms “heteroazeotrope” and “heterogeneous azeotrope” include an azeotrope-like compositions comprising a vapor phase existing concurrently with two liquid phases.

In some embodiments, methods of cleaning reactors or separating (E)-1-chloro-3,3,3-trifluoropropene, HF, and a heavy organic may include forming an azeotropic or azeotrope-like composition. The azeotropic or azeotrope-like composition may include an azeotrope between HF and at least one of 240fa, 241fa, 242fa, or combinations thereof.

For example, azeotropes of HF and 241fa may as little as about 2 wt. % HF, 15 wt. %, 30 wt. %, 50% wt. % HF, as great as 60 wt. % HF, 70 wt. % HF, 90 wt. % HF, and 99 wt. % HF or within any range defined between any two of the foregoing values (such as between about 31 wt. % HF and about 72 wt. % HF, between about 2 wt. % HF to about 99 wt. % HF, and between about 15 wt. % to about 90 wt. %). Furthermore, in one example, a heterogeneous azeotrope was found to have 2 wt. % 241fa and 98 wt. % HF in a vapor stream, with a top liquid layer having 15 wt. % 241fa and 85% HF, and a bottom liquid layer of 99 wt. % 241fa and 1 wt. % HF.

Also, azeotropic or azeotrope-like mixtures of 1233zd(E) and HF may be formed. In some embodiments, the azeotropic or azeotrope-like mixture of 1233zd(E) and HF has a boiling point of about 0 to about 60° C. at a pressure of about 3 psia to about 73 psia.

The embodiments or examples disclosed below are not intended to be exhaustive or limit the disclosure to the precise form disclosed in the following detailed description. Rather, the embodiments are chosen and described so that others skilled in the art may utilize their teachings.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. § 112(f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A method of separating heavy organics from a mixture of heavy organics, HF, and light organics, the light organics including (E)-1-chloro-3,3,3-trifluoropropene, the method comprising:
separating the mixture into an HF stream and an organics stream, the HF stream comprising HF and some of the light organics, and the organics stream comprising the heavy organics and the balance of the light organics; and
separating the HF from the light organics in the HF stream.

2. The method of claim 1, further including providing the mixture from a reactor configured to produce (E)-1-chloro-3,3,3-trifluoropropene.

3. The method of claim 1, wherein separating the mixture into an HF stream and an organics stream comprises at least one of decanting, centrifuging, liquid-liquid extracting, distilling, flash distilling, or combinations thereof.

4. The method of claim 1, wherein separating the mixture into an HF stream and an organics stream consists of flash distillation.

5. The method of claim 1, wherein separating the mixture into an HF stream and an organics stream consists of liquid-liquid separation in which the HF stream is an HF phase and the organics stream is an organics phase.

6. The method of claim 1, further including distilling the organics stream to separate the light organics from the heavy organics.

7. The method of claim 6, further including disposing of the heavy organics.

8. The method of claim 6, further including recovering the heavy organics.

9. The method of claim 1, wherein separating the HF from the light organics in the HF stream comprises distilling the HF stream.

10. The method of claim 1, wherein the heavy organics have a weight average (Mw) molecular weight between about 500 g/mol to about 7,000 g/mol.

11. The method of claim 1, wherein the heavy organics have a boiling point between about 120° C. and about 300° C. at a pressure between about 3 psia to about 73 psia.

12. A method for removing heavy organics from a reactor configured to produce (E)-1-chloro-3,3,3-trifluoropropene, the method comprising:
purging from the reactor a stream comprising the heavy organics, HF, and light organics, the light organics including (E)-1-chloro-3,3,3-trifluoropropene; and
separating the purge stream into an HF stream and an organics stream, the HF stream comprising HF and some of the light organics, the organics stream comprising the heavy organics and the balance of the light organics.

13. The method of claim 12, further including distilling the HF stream to separate the HF from the light organics.

14. The method of claim 13, further including recycling the HF from the HF stream back to the reactor.

15. The method of claim 14, further including recycling the light organics from the HF stream back to the reactor.

16. The method of claim 14, further including distilling the organics stream to separate the heavy organics from light organics.

17. The method of claim 15, further including recycling the light organics from the organics stream back to the reactor.

18. The method of claim 12, wherein separating the purge stream into an HF stream and an organics stream comprises at least one of decanting, centrifuging, liquid-liquid separation, distilling, flash distilling, or combinations thereof.

19. The method of claim 12, wherein separating the purge stream into an HF stream and an organics stream consists of flash distillation.

20. The method of claim 12, wherein separating the purge stream into an HF stream and an organics stream consists of liquid-liquid separation in which the HF stream is an HF phase and the organics stream is an organics phase.
